(19) United States
(12) Patent Application Publication
(10) Pub. No.: US 2012/0308963 A1
(43) Pub. Date: Dec. 6, 2012

(54) DENTAL DIAGNOSIC AND DENTAL RESTORATION METHODS, SYSTEMS, APPARATUSES, AND DEVICES

(75) Inventors: Gunnar Hasselgren, Tenafly, NJ (US); Anas Selman, Davie, FL (US); Chia-Yi Chen, New Hyde Park, NY (US)

(73) Assignee: The Trustees of Columbia University in the City of New York, New York, NY (US)

(21) Appl. No.: 13/499,620

(22) PCT Filed: Sep. 30, 2010
(86) PCT No.: PCT/US2010/050887

§ 371 Date: Aug. 13, 2012

Related U.S. Application Data
(60) Provisional application No. 61/247,689, filed on Oct. 1, 2009.
(60) Provisional application No. 61/247,726, filed on Oct. 1, 2009.

(51) Int. Cl. A61C 8/00; A61B 6/14; G06F 17/50; A61B 6/08

(52) U.S. Cl. 433/201.1; 378/205; 700/98

(57) ABSTRACT

Devices, methods, and systems for the design, fabrication, modification, and implantation of dental prostheses. An implant is created that has a shape closely or precisely conforming to the natural shape of a modified or unmodified tooth socket. The implant is used to anchor any of a variety of dental restorations or other substitute devices to bone. Further, methods, systems, and apparatuses for taking radiographs at specific, standard, and/or reproducible angles. A first radiograph is taken, using an aiming apparatus, with the aiming direction forming a first angle with the normal to an image plane of an image receptor. At least one additional radiograph is taken, using the aiming apparatus, at a different aiming direction from the first direction. Radiographs taken with the multiple aiming angles are used to create a three-dimensional image of an object represented by the radiographs.

### CROSS REFERENCE TO RELATED APPLICATIONS

The present application is an International application, which claims the benefit of U.S. Provisional Application No. 61/247,689, filed Oct. 1, 2009, and U.S. Provisional Application No. 61/247,726, filed Oct. 1, 2009, both of which are incorporated herein by reference in their entireties.

### BACKGROUND

This application relates to dental restorations and particularly to dental implants, methods, and systems which appear and/or function as a natural root of a tooth. This application also relates to related methods, devices, systems, and articles of manufacture including radiographic aiming devices.

Dental implants have proven to be an effective device for restoring lost function in patients with missing teeth. Implants provide foundation upon which a prosthetic tooth, or teeth, or other restoration, such as a bridge, can be secured. This application also relates to dental radiographs. Radiographs, two-dimensional images of three-dimensional objects produced using X-ray technology, are essential to most phases of dental therapy and diagnosis. In the dental realm, radiographs are produced when X-rays pass through an object or objects (e.g., a tooth, a tooth root, and/or surrounding structure) and interact with an image receptor, such as photographic film or a digital sensor positioned in a patient's mouth behind the object, or objects, to be imaged. Such radiographs may be used to inform a diagnosis at various treatment phases or to help evaluate the success or failure of a treatment (e.g., an endodontic treatment). In addition to diagnosis, radiographs are used to determine, anatomic landmarks, canal working lengths, master cone adaptation, the quality of the root canal filling, the location of canals, or superimposed objects, such as teeth, roots, canals, etc.

In producing radiographs, many practitioners use a positioning instrument to assist in aiming the X-ray beams output from a tube head of an X-ray apparatus toward the object and image receptor. This practice has become the standard of care. Typically, the tube head of the X-ray apparatus is aligned such that the outputted X-rays are orthogonal to, or substantially orthogonal to, the image receptor.

### SUMMARY

Disclosed embodiments relate to devices, methods, and systems for the design, fabrication, modification, and implantation of dental prostheses that replace natural teeth which have been extracted, or natural teeth or roots thereof that are scheduled for extraction. In embodiments, an implant has a shape that closely, or precisely, conforms to the natural shape of the tooth socket. The implants can be used to anchor any of a variety of dental restorations or other substitute devices to bone, particularly tooth implants and bridge implants as well as removable dentures and other devices.

Embodiments include an implant shaped to conform to the existing shape of a tooth socket or the socket that supports or supported a root with a furcation. In such embodiments, the implant may be shaped such that furcation in the implant is eliminated. The socket may be modified to remove tissue that had lain in the furcation. The implant may have an external surface which, along with the general natural shape, promotes bio-integration. For example, it may be cast from titanium or an alloy thereof in such a manner as to generate a rough surface.

The shape of the natural root or socket can be modeled based on multiple radiographic images of the root and/or socket, using computational reconstruction of the socket surface. For example, the planar projections of the root created by multiple X-rays taken based on multiple aiming directions may be combined with predictive information about possible anatomical shapes, to create a unique model of an accommodating root shape. Alternatively, computer aided tomography (CAT) or magnetic resonance imaging (MRI), or any other suitable technique for acquiring three-dimensional structural information can be used.

The implant model may be computationally adjusted based on a prediction of an amount of periodontal ligament to remain in the socket after root extraction, for example, by contracting the implant model from the dimensions of the bone by a predefined distance or by imaging the soft tissue. Full or partial removal of the periodontal ligament may also be provided in a suitable method. The model of the implant may be rendered into an implant by any suitable means. In a particular embodiment, a wax cast is machined and a mold created from the machined part to create a titanium (or alloy of titanium) casting whose surface roughness is well adapted to biointegration.

A disclosed embodiment includes a method of making a dental implant, including creating a three-dimensional model of a furcated natural tooth root and/or socket based on imagery from a patient's unique anatomy and increasing a volume of the model the at the expense of volume between the furcations whereby the depth of the furcations is reduced relative to the natural root or socket. In this embodiment, the model is faithful, other than the reduction of the furcation, to the shape of the natural tooth or socket. The method may further include forming an implant from the model. The casting may be of titanium or titanium alloy or any biocompatible material including ceramic, polymer, or a non-biocompatible material coated with a biocompatible material. The forming may include casting or machining or both. In an embodiment, a wax form is machined from the model using computer aided manufacturing (CAM) techniques and a mold made from the wax form. An implant is then cast from the mold. In another embodiment, an implant is machined directly. In yet another embodiment, the implant is made by three-dimensional printing, photopolymerization, lithographic techniques or any other suitable method.

In an embodiment where the target socket contains, or contained, a furcated root, the recess between the furcations from the model is eliminated entirely from the model, by filling in the recess completely, prior to fabrication of the implant. In another embodiment, the elimination of the recess results in an implant in which the surface thereof, over a portion lying below the bone line after implantation, is a convex surface (i.e., one with no concave portions). In the above embodiments, the model may be a numerical model, such as typically applied in computer aided design (CAD). For example, a target tooth or socket can be modeled as a triangular mesh representing the tooth surface.

In any of the embodiments, a portion of the periodontal ligament can be retained in the socket and the model is modified to accommodate it. For example, a predicted reduction of the size of the implant model is made to account for the thickness of the ligament. In any of the embodiments, the implant is formed from the model such that when implanted, except for the access to the socket, it is entirely encased in bone. As such, after implantation, bone may grow over the top of the implant.

In any of the embodiments, an implant system includes an abutment that can be attached to the implant and to which a crown or bridge or other restoration or prosthesis can be attached. The separate abutment permits the relationship between the restoration or other prosthesis, such as a crown, to be modified, as desired, to provide a conforming or otherwise desired fit with affected anatomy adjacent thereto or abutting the same. In any of the embodiments, the implant may be machined to create features to permit the attachment of the abutment. For example, the implant may be machined to form a supporting surface such as a recess into which a mating feature, such as a post, on the abutment fits. In addition, a threaded recess may be formed in the implant by drilling and tapping. In a feature, applicable to any of the embodiments, the abutment is attached, by a bolt, to the implant. In a method of using the abutment, the abutment is formed after the implantation and healing of the implant. Impression molding, CAD modeling, and/or other techniques are then applied to the fabrication of an abutment that causes the restoration to provide a desired relationship to the affected anatomy; i.e., any anatomy that is visually or mechanically impacted by it.

In any of the embodiments disclosed herein, the implant model may be derived from a non-furcated root and all the embodiment features described above may be applied thereto, except those relating to furcated roots or sockets. In an embodiment of an implant that replaces a tooth whose root lies close to the buccal side surface of bone, such as an incisor, an implant is shaped such that a larger amount of bone lies between outside surface of the bone and the implant. The final model shape may be obtained by eroding the root model of the implant that faces the outside and replacing it with a portion of a material such as one that promotes osteogenesis, such as hydroxyapatite. The shape of the implant may further depart from the natural socket by providing surface features (standoffs) that help to hold the implant in position while promoting the growth of bone into the space between the implant and the natural socket.

Embodiments include a three-part restoration system including a prosthetic element that may be a bridge, a crown, or other prosthetic. The restoration system includes an implant and a joining abutment. The abutment is configured to be removably attachable to the implant. The implant can be a conventional implant, modified to make it compatible with the abutment, or a naturally-shaped implant according to any of the embodiments disclosed herein. The abutment may be attachable to the implant by a screw or a nut or other mechanical locking mechanism. The abutment may also be attachable by a removable adhesive. The prosthetic element may be removably attachable to the abutment. A cement may be used to attach the prosthetic element to the abutment, where the cement can be dissolved or otherwise disintegrated to permit a screw used to attach the abutment to be engaged, thereby permitting replacement of the abutment.

In a method of using the three-part restoration system, an implant is fabricated according to any of the disclosed embodiments and implanted. After a healing period, for example, three weeks or more, an abutment and a restoration element are fabricated using CAD or impression-based techniques or a combination thereof. These fabrication techniques may be used to provide a custom fit to the patient's anatomy. Alternatively, the restoration element and/or abutment may be prefabricated and form members of a kit having a range of alternative sizes and shapes permitting a selected one to be chosen as a best-fit to the patient's current or predicted anatomy—the term “anatomy” being used herein to identify any artificial or natural anatomical features. The method of use may further include the replacement of the abutment, and/or restoration, at a later time to improve the fit with the anatomy, for example after a period of time in which a shift occurs due to other changes in anatomy resulting from surgery or natural changes due to wear, aging, growth, or other reasons.

Another method is for the making of a dental implant. The method may comprise fabricating a model of a natural furcated root and forming, responsively to the model, an implant whose sides are shaped substantially as the natural root but with an end that has a shallower recess between the furcations than the natural furcated root. The model may be a faithful copy of the root of a tooth whose extraction is planned. The model may alternatively be a faithful copy, or a prediction derived from an image, of a natural tooth socket. The fabricating may include generating a numerical model of the implant and adding volume to the model such as to reduce the depth of the recess between the furcations. The fabricating may include generating a numerical model of the implant and adding volume to the model such as to eliminate the recess between the furcations. The fabricating may include forming a model with a convex surface where a concave surface exists in a copied natural root.

In any of the embodiments, the implant and socket may be modified by fabricating mating features into them to facilitate short or long-term anchoring of the implant. For example, the implant may have a recess or protrusion and the socket may be modified, such as by machining, to create a mating protrusion or recess that fits into or receives a mating recess or implant of the implant. In addition, a protrusion may be formed in the implant by a screw.

The disclosed subject matter also relates generally to dental radiographic imaging. Among features of the disclosed subject matter is the procurement of radiographs using specific, standard, and/or reproducible angles. It may be desirable to provide one or more additional, “supplemental” radiographs from perspective angles different from the angle of the first taken radiograph (e.g., the orthogonal radiograph). These supplemental radiographs can enhance visualization and evaluation of the three-dimensional structure of the object or objects. Radiographs taken at specific, standard, reproducible angles can lead to an increase in patient safety because the number of radiograph re-takes will decrease, thereby exposing the patient to less radiation during the imaging process. An aiming apparatus can be used to obtain the specific, standard, and/or reproducible angles. In embodiments, the angles may be chosen to provide satisfactory image information to allow computation of a representative three-dimensional mathematical model of a target, such as a tooth. In this regard, a predicted range of shapes of the target can allow such three-dimensional models to be derived from a small number of images, for example, two.

According to an embodiment, the disclosed subject matter includes a method, apparatus, and/or system for taking radiographs. A first radiograph is taken with the aiming direction forming a first angle with the normal to an image plane of the image receptor. For example, the aiming direction may be parallel to the normal of the plane of the image receptor (orthogonal angle). A second radiograph may be taken with a different aiming direction. The angular space between the aiming directions can range from ten degrees to twenty-two degrees. The radiograph taken with the multiple aiming angles may be used to create a three-dimensional image of an object represented by the radiographs using techniques such as described in U.S. Pat. Nos. 6,816,564 and 6,049,582 incorporated by reference in their entireties. The aiming directions can be equally spaced from the normal to the plane of the image receptor. Alternatively, one aiming direction can be aligned with the normal and one can be oblique to the normal of a planar image receiver. In an embodiment, the angle (or angles) between the aiming directions may lie in a plane that is approximately normal to a tooth root axis. In another embodiment, the angular separation between multiple aiming directions may include ones that lie in the plane that is approximately normal to a tooth root axis and components that lie in a plane that is oblique or parallel to a tooth root axis,

In another embodiment, a multiple ring aiming device, where the rings define aiming angles separated by from about ten to twenty-two degrees, has a connector that allows it to be connected to a bitewing bite block that supports a radiographic imaging device. The multiple ring aiming device may have two rings. In another example, the aiming device can have three rings with two spaced by a maximum angular separation and one located between the two, separated from each by an angle less than the maximum separation. In embodiments, the rings may form an integral structure.

In all the aiming device embodiments, the aiming device may be provided with a support member adapted to fixedly position and orient the aiming device with respect to dental anatomy of a patient. For example, the aiming rings may be provided with a support boss with a hole. The hole may be shaped to receive a support arm extending from a bitewing bite block or other type of support. For example the hole may have a polygonal or other shape for receiving a support arm with a similar shape. The polygon or other shape may be selected to secure against undesired rotation about the support arm axis. In an alternative embodiment, instead of a single multiple-ring device, a kit of rings, each defining different aiming angles with respect to the support arm, and thereby the target anatomy, is provided. Each of the rings defines an angle, with reference to the arm, such that the aiming directions are separated by the angles predetermined to be suitable for forming, when synthesized using a computer, a three-dimensional model of the target tooth.

### BRIEF DESCRIPTION OF THE DRAWINGS

The accompanying drawings illustrate embodiments of the disclosed subject matter. The disclosed subject matter will be best understood by reading the ensuing specification in conjunction with the drawing figures, in which like elements are designated by like reference numerals, and wherein:

FIGS. 1A and 1B illustrate a procedure for creation and placement of a dental prosthesis according to embodiments of the disclosed subject matter.

FIG. 1C shows a finished implant according to embodiments of the disclosed subject matter.

FIG. 1D is an illustration of a socket with tissue at the base of the socket, possibly including alveolar bone, removed to accommodate an implant according to embodiments of the disclosed subject matter.

FIG. 2A is an exploded view of the parts of dental prosthesis according to embodiments of the disclosed subject matter.

FIG. 2B is an assembled view of a dental prosthesis according to embodiments of the disclosed subject matter.

FIG. 3 illustrates a bridge affixed by implants according to embodiments of the disclosed subject matter.

FIG. 4A is an illustration of a socket to accommodate an implant according to embodiments of the disclosed subject matter.

FIG. 4B is an illustration of a socket to accommodate an implant having a concavity at a bottom thereof according to embodiments of the disclosed subject matter.

FIG. 4C is an illustration of a socket to accommodate a convex implant according to embodiments of the disclosed subject matter.

FIG. 4D shows a finished implant according to embodiments of the disclosed subject matter.

FIG. 5A shows an incisor in section.

FIG. 5B shows, in partial section, an implant/dental prosthesis for use where the thickness of the bone supporting the implant is relatively thin according to embodiments of the disclosed subject matter.

FIGS. 6A, 6B, 6C, and 6D illustrate an implant/dental prosthesis for use where the thickness of the bone supporting the implant is relatively thin according to further embodiments of the disclosed subject matter.

FIG. 7 illustrates an implant with a through-hole for anchoring the implant according to embodiments of the disclosed subject matter.

FIG. 8A is a perspective view of an aiming apparatus which may be used as part of a radiographic system according to embodiments of the disclosed subject matter.

FIG. 8B is a plan view of the aiming apparatus of FIG. 8A coupled in a first position to a bitewing image receptor support, the combination of which may be used with a radiographic imagine system according to embodiments of the disclosed subject matter.

FIG. 8C is a plan view of the aiming apparatus of FIG. 8A coupled in a second position to a bitewing image receptor support, the combination of which may be used with a radiographic imagine system according to embodiments of the disclosed subject matter.

FIG. 9 is a plan view of a multi-direction aiming apparatus similar to the embodiment of FIGS. 8B and 8C defining three aiming directions.

FIG. 10 is a perspective view of a multi-direction aiming device similar to the device of FIG. 8A and having multiple positioning bosses.

FIGS. 11A-11D illustrate an aiming apparatus for a radiographic system according to embodiments of the disclosed subject matter.

FIGS. 12A and 12B illustrate members of a kit of single-ring aiming apparatuses according to embodiments of the disclosed subject matter.

### DETAILED DESCRIPTION

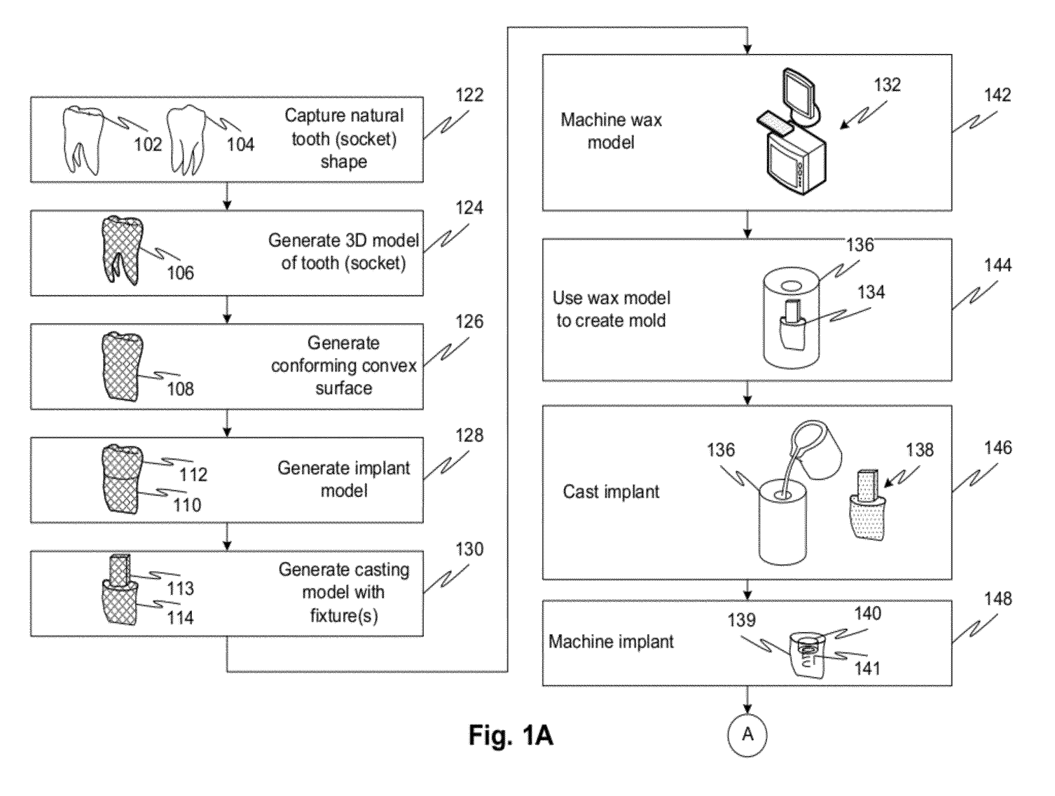

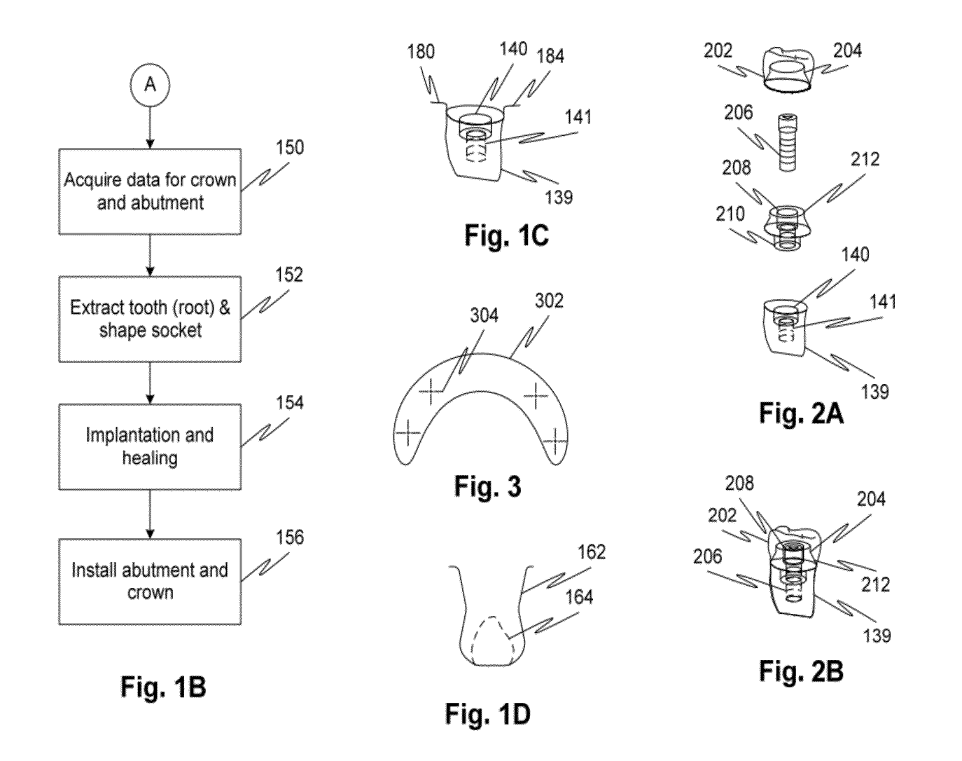

FIGS. 1A and 1B illustrate a procedure for creating and placing a dental prosthesis. A patient may visit a dental facility for a dental evaluation by a dental clinician. During the evaluation, the clinician may take one or more radiographs of the patient's teeth and surrounding structure. Examination of the radiographs can identify problems with one or more of the patient's teeth and/or surrounding structure. In certain instances, the problems may require removal of one or more of the patient's teeth. If a tooth is not replaced after extraction, one or more of the patient's remaining teeth may move because of the additional space. Such movement can lead to problems, such as bite alteration and speech issues. An extracted tooth and/or root may be replaced by a dental prosthesis to fill the void left by the extracted tooth and/or root and replace the function of the extracted or missing tooth.

Upon determination by a dental clinician that a tooth is to be extracted and replaced with a dental prosthesis, data representing the tooth, tooth root, and/or surrounding structure, such as the socket and bone shape, may be acquired 122. For example, the target anatomy may be scanned via radiographic three-dimensional imaging prior to extraction, or multiple planar images may be used to synthesize a three-dimensional model of the target surface or surfaces as indicated at 124. One or more two-dimensional X-ray images (planar projections onto an imaging device) can be taken, for instance.

Alternatives that can be used in all examples of radiographic acquisition of a tooth model, according to embodiments of the disclosed subject matter, include optical coherence interferometry, ultrasound, terahertz wave imaging, and extraction and mechanical or laser scanning. Many different techniques may be used in acquiring a faithful model of a tooth or other anatomy and it will be clear from the present disclosure that such other techniques may be used in conjunction with various features of the disclosed embodiments.

Ultimately, the target representation information acquired at 122 is used to create and place a dental prosthesis including an implant, a crown, and an optional abutment. At this time, data that is only used for creating and/or placing the implant may be obtained. However, data for creating and/or placement of an abutment and/or a crown may also be acquired at this stage as well. Imaging can be used to acquire structure of the surrounding tissue, such as bone tissue between tooth roots in the case of a tooth having multiple, furcated roots. The illustration indicated at 122 shows two views 102 and 104 of a target tooth, each taken from a different perspective (e.g., aiming angle). Other capture techniques may be used rather than digital reconstruction of a three-dimensional shape from planar projections at multiple angles. Radiography methods include tomography techniques, computed tomography (“CT”) techniques, and cone beam CT techniques. MRI techniques may also be used. Impression techniques may also be used. Other devices and techniques may also be used such as optical coherence interferometry, ultrasound, terahertz wave imaging, and extraction and mechanical or laser scanning. Combinations of any of the above techniques may be used.

CT scanners use X-rays to produce sectional or slice images, as in conventional tomography, but the radiograph film is replaced by very sensitive crystal or gas detectors. The detectors measure the intensity of the X-ray beam emerging from the patient and convert this into digital data which can be stored and manipulated by a computer. This numerical information is converted into grey scale representing different tissue densities, thus allowing a visual image to be generated. Cone beam CT (or digital volume tomography), which involves low dose cone beam CT technology, employs a cone-shaped X-ray beam, rather than the flat fan-shaped beam used in conventional CT, and a special detector (e.g., an image intensifier or an amorphous silicon flat panel). The equipment orbits around the patient, taking approximately 20-40 seconds, and in one cycle or scan, images a cylindrical or spherical volume—described as field of view.

With the tomography images acquired, a three-dimensional representation or model 106 of the tooth and/or tooth root can be generated 124. Impressions also can be used to obtain a representation of the tooth and/or surrounding structure (e.g., adjacent teeth) for creating the three-dimensional model. A three-dimensional model 106 of a tooth having three roots, for example, may be generated. The three-dimensional model 106 can be generated using computer software. For example, computer aided design (“CAD”) or computer-aided design and drafting (“CADD”) software can be used to create the model or models 106, with the tomography images being transferred into a standard CAD/CADD file, and the CAD/CADD software recognizing the tooth as a separate structure from the surrounding bone/tissue.

If the tooth has multiple or furcated roots, computer software can modify the initial “natural” three-dimensional image or model 106 of the tooth and tooth roots to generate a modified image or model 108 at 126. For example, the volume between the roots (occupied by bone tissue, for example) can be fully or partially “filled-in.” Note that as used herein, the description “adding volume” may refer to surface modification such as reducing a depth of a concavity of a surface portion of a surface model of an implant or other structure and is not intended to limit embodiments to the manipulation of models that define volumes, as such, for example as constellations of voxels.

The modifying may include the creation of a conforming convex surface such as might be illustrated by the stretching of a fabric over three dimensional object with concave surfaces. By filling in, fully or partially, the volume between the roots in a furcated root setting, model 108 with a non-furcated root structure can be created. Put another way, the three dimensional model or template 108 is created based on a furcated natural tooth root having a recess between the branches of the root furcation, wherein the computer software reduces a depth of the recess by adding volume to the template. As shown in 126 in FIG. 1A, the resultant model 108 shows a fully filled-in volume between the roots, resulting in a model with non-furcated root structure. Various algorithms may be applied for the filling in of furcations and other modifications of the model, such as a surface spline fit with a minimum smoothness constraint.

Dental implants may be used to replace teeth with or without a furcated root structure. Furcations in regular teeth can become problems in older patients, for example, when the gingiva (gum tissue) has receded and the split or furcation between the roots becomes an area that is very difficult to keep clean. Bacteria can hide in this recessed portion. Peri-implantitis, an inflammation surrounding an implant, can occur and the implant can fail. The minimization of the furcation may be based on amelioration of this problem by modifying the furcation(s) by reducing concavities to predefined depths or enforcing a smoothness threshold, each of which is chosen to prevent the problem of trapped bacteria.

Similarly, if the root structure or geography of adjacent bone tissue for a tooth having one or more roots has a unique characteristic, such as a recessed portion or other void (e.g., a void between root structure and adjacent bone tissue due to recession of tissue), the CAD software can modify the natural three-dimensional model of the tooth and/or tooth root(s). For example, some or all of the volume of the recessed portion or void can be filled-in to create a modified image or model.

Note that automated algorithms, user-controlled editing tools, and combinations thereof may be employed in making the modifications.

Based on the imaging and three-dimensional model, a three-dimensional image or model of a portion of the dental prosthesis is generated 128. The model or image can be created using computer software, and the portion of the dental prosthesis created at this point can be the implant 110, an abutment for coupling to the implant (not explicitly shown), and/or a crown 112 for coupling to the abutment. FIG. 1A, for example, shows a model of the dental prosthesis with the implant portion 110, abutment portion (not shown), and the crown portion 112. Software may calculate the load applied to one or more portions of the prosthesis during different jaw movements, for example. Such calculations can aid in designing the one or more portions of the prosthesis to obtain optimal stress distribution.

After creation of the implant model, the implant is fabricated using any suitable technique. At the same time, a corresponding abutment and crown also can be created. The implant can be created in a lab or at a treatment location. For example, data representing the three-dimensional model of the implant can be transmitted to a lab and used to fabricate the implant which can then be shipped to the clinician at a treatment location. FIG. 1A, at 130-146, illustrates an embodiment in which a casting technique is used to fabricate the implant. Casting can provide for a customized fit, which theoretically leads to better implant-to-bone integration (osseointegration), as well as fewer infections and complications.

A casting model 114 of the implant is created in software at 130. The casting model 114 can have a handle 113, which can be used for handling of the casting model 114 and which may be removed by machining.

The casting can use the lost wax method. The lost wax method includes making a wax model using, for example, computer aided manufacturing (“CAM”) 132 to mill the wax model at 142. The wax model 136 is used to create a mold 134 for casting the implant at 144. Though not shown, threading for connecting an abutment or crown-restoration to the implant can be provided for by the wax model 136 using a casting technique rather than subsequent machining as described in the present embodiment.

A material is cast in the mold 134 of the model 136 at 146. A desirable material is pure titanium or alloy thereof that has been heated to liquefaction in an oxygen-free environment by induction heating, for example, wherein the oxygen-free environment may be created by purging with an inert gas such as argon gas.

In various embodiments, the cast implant 138 may be inspected for imperfections after the casting. Though not explicitly shown in the casting at 146, the implant may be cast in a ready-to-place form, with no additional milling or other physical alterations necessary before placement. Features to permit the attachment of an abutment and/or a crown can be provided by the wax model, such as a supporting surface and/or a threaded recess. The implant is then sterilized and made ready for placement in a socket, for example, the socket of a newly extracted tooth.

If a handle 113 is provided, it may be removed before preparation of a receiving portion configured to mate to an abutment and/or a crown. After the implant 139 has been cast at 146, the implant 139 may be machined to create features to permit the attachment of an abutment and/or crown at 148. For example, the implant 139 may be machined to form a supporting surface such as a recess 140 which may be a socket shaped feature. In addition, a threaded recess 141 may formed by drilling and tapping. In a feature, applicable to any of the embodiments, the abutment is attached, by a bolt or screw, to the implant 139.

The implant 139 also can be created by milling using a milling apparatus based on a milling model (not shown). The milling apparatus can perform the milling by a number of methods, such as laser milling, cutting, grinding, chemical etching, or other techniques. In addition, techniques such as three-dimensional printing lithography, or photopolymerization can be used to create a degradable model that can be used to create a mold. Note that as stated elsewhere, an intermediate wax model with casting is only one alternative and other fabrication techniques may be used.

In various embodiments, after the milling, the implant can have rough surfaces. These rough surfaces can be smoothed by machining, tumbling, or etching. Optionally, these rough surfaces can be left in place since rough surfaces can contribute to strength of the integration of the implant and with surrounding tissue, for example bone.

The implant 139, whether milled or cast, can also be provided with surface augmentation features, such as studs, pits, striations, etc. These may be provided at the model generation phase 128 or at another stage by suitable additional fabrication steps. In embodiments, the implant 139 may have prepared channels formed therein, wherein the channels may be perpendicular to the long axis of the implant and close to the apex, away from the oral cavity. Such surface augmentation can be beneficial for bone growth into, on, and around the surface augmentations. The surface augmentation also may assist with retention of the implant 139 in the socket. Another type of augmentation may be the provision of one or more through-holes as described below with reference to FIG. 7 to allow bone to grow deeply into the implant.

The patient may return to the clinician's office for a second visit, at which time, if not acquired at a prior visit, or in addition to data acquired at a previous visit (e.g., when creating the implant model), data for creation and/or placement of a suitable crown and/or abutment may be collected 150. Such data may include various patient anatomical information, such as adjacent tooth position, spacing, etc.

After creation of the implant 139, a tooth or tooth root may be removed and the implant emplaced in the socket 152-154. Optionally, some or all of the remaining periodontal ligaments can be removed. Alternatively, some or all of the remaining periodontal ligaments can be left. If some or all of the periodontal ligament(s) is/are removed, the dimensions of the wax model may be adjusted to compensate. For example, a space of 0.15 mm may have to be provided in the wax model generated at 124.

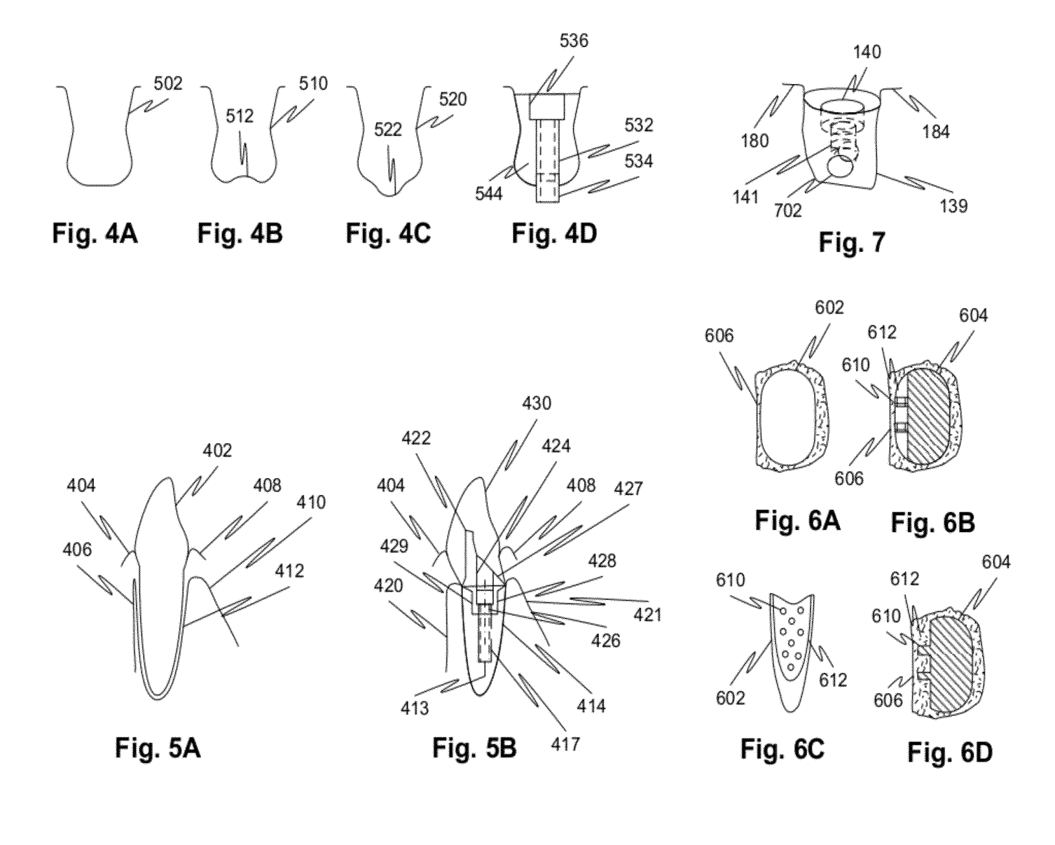

Referring to FIG. 1D, for a tooth with furcated roots, before placement of the implant 139 in the socket, the alveolar bone portion 164 filling the space between the roots can be shaped or otherwise modified to create a modified tooth socket 162. The socket 162 may be “shaped” to conform to the implant 139 to be placed. FIG. 1D shows an alveolar bone 164 and surrounding tissue between a furcated root structure that may be removed. Some or all of this bone tissue may be removed before placement of the implant. A special tool may be used to remove some or all of the bone, such as a rongeur, bone forceps, a drill, etc. The special tool can be configured to modify the socket based on the configuration of the implant to be placed therein. Furthermore, the special tool can be used to create a precise interface for the bottom of the socket and the bottom of the implant. For example, the bottom of the implant to be inserted may be substantially flat for a socket bottom that has been modified or is otherwise substantially flat, such as shown in FIG. 4A. The special tool also can be used to create a support feature in the socket, preferably at the bottom.

FIGS. 4A-C show examples of manners in which the socket can be shaped or modified to mate with a conforming shape of a modified implant. FIG. 4A is similar to FIG. 1D, with the entire alveolar bone portion having been removed from socket 502. FIG. 4B shows socket 510 with a small portion of the alveolar bone remaining in the form of a protrusion or a stub 512. The portion of the alveolar bone left remaining may be used to anchor or otherwise retain an implant with a concave bottom portion to the socket 510. In FIG. 4C, the entire alveolar bone portion has been removed from the socket 520, and a portion of the bone tissue below has been removed, creating a recess 522. This recessed portion 522 in the bone tissue may be used to anchor an implant with a convex bottom portion to the socket 520. In FIG. 4D, a retaining member 534 extends into the recessed portion in the bone tissue for anchoring the implant 544. The retaining member 534 may be a stud or screw that is pushed or threaded through a hole 532 in an implant 544. A recess that mates with an abutment is indicated at 536.

After the tooth has been removed and the socket shaped at 152, if necessary, the implant 139 may be emplaced 154. Data for creating and positioning of an abutment and/or a crown can be obtained at this time (e.g., additional imaging data). From the data taken with the implant and healing, if any, an abutment and/or crown can be modeled and created. Alternatively, depending on the clinical situation, the implant 139 can be directly loaded with a crown, temporary or permanent. Alternatively, the implant 139 may be emplaced, including being enclosed by overlying soft tissue, for a healing interval 154. The implant 139 may be fabricated to lie below the bone line such that bone is permitted to grow over the top of the implant 139. As can be seen from FIG. 1C, an implant 139 is arranged below the bone line 180, 184. The implant 139 may be made so that a top surface thereof, or a part of the top surface, lies below the bone line. This may enhance the strength of the implant and also help in healing because the implant 139 can be more easily covered (temporarily) by soft tissue. The implant may be sized so that when healing occurs, the top is partially covered by bone. Before attaching the abutment, the top portion of the implant 139 may be covered partially or completely by soft tissue to assist with healing.

If the implant is covered by soft tissue during healing, wherein only the implant 139 is emplaced, the implant 139 later may be permanently restored with a crown that is connected to the implant via an abutment with a screw 206. The abutment may be fabricated or selected from a kit after the healing interval.

FIGS. 2A and 2B show exploded and assembled views of a dental prosthesis according to embodiments of the disclosed subject matter. The dental prosthesis includes an implant 139, an abutment 212, and a crown 202. The implant 139 includes a receiving opening or recess 140 to receive a mating feature 210 of the abutment 212. The implant 139 also has a threaded recess 142 to receive a screw 206 used to secure the abutment 212 to the implant 139. The abutment 212 may include a recess 208 into which the head of the screw 206 is recessed and upon a blind end of which the screw 206 head exerts a binding force. The crown 202 may have a recess 204 that is configured to fit closely the abutment 212. The crown 202 and abutment 212 may be configured for a snap-fit.

FIG. 3 illustrates a restoration including a bridge 302 that may be affixed to abutments located at various points such as indicated at 304. The abutments in this case may be attached as described elsewhere herein, to implants as also described herein.

Existing presentment screw-type dental implants are factory machined with screws for placement into the alveolar bone (bone of jaw). Presentment screw-type implants generally need to be placed in “solid bone,” where the bone is most dense, and permitted to osseointegrate. In the front region of the mouth, placement of conventional screw-type implants can cause aesthetic and/or functional problems. In particular, because of the thin facial bone structure (the bone on the buccal side of the tooth), such presentment screw type-implants can cause problems when placed close to the facial surface of the bone (e.g., in the upper front tooth region). Instead they must be placed further into the oral cavity, which makes restoration difficult both functionally and esthetically. Screwing the implant into the thin facial bone surface can crack the facial thin bone surface such that screw threads project from the bone surface. Such cracking can impair or prevent bone formation and ultimately lead to implant failure.

FIG. 5A shows an incisor in section and FIG. 5B shows, in partial section, an incisor prosthesis according to embodiments of the disclosed subject matter. A natural tooth 402 is supported by bone 410 and 406 of the jaw. A space where the natural periodontal ligament resides is also shown at 412. The lingual 408 and buccal 404 surfaces of the adjacent gum are also indicated. The bone 406 forming part of the jaw on the buccal side is thinner than the lingual side 410. The features of FIG. 5A are shown for the example of an incisor, but it is possible for one portion of bone supporting a tooth to be thinner on one side than on another under other conditions, so the incisor is described merely as an example.

As shown in FIG. 5B, a restoration is placed in the bone in a more lingual position than the original socket such that a thicker layer of bone 420 on the buccal side is formed. The new socket may be formed by reshaping the socket by machining and filling in a portion with bone, for example, by filling with a temporary implant with a porous element that integrates with bone such as metal sponge or coral. Promoters of osteogenesis such as hydroxyapatite may be used. Alternatively the socket may be filled-in completely with bone and a new socket formed by machining.

FIG. 5B illustrates a completed restoration with supporting bone 420 and 421 with a socket 414 into which an implant 413 has been implanted. The implant 413 has a recess 428 which may be formed by machining along with a tapped threaded hole 417 with a screw 426 threaded thereinto. An abutment 427 is shaped to fit closely in the recess 428 and therefore has a shaft 429 portion that extends into the recess 428. The abutment 427 has a dome 422 to which the crown 430 is attached, for example by adhesive or cement. The dome 422 is offset relative to the socket axis to place the crown 430 in a natural position with respect to the affected anatomy and gum surfaces 404 and 408. A hole 424 provides access for a tool to a head of the screw 426.

The abutment 427 is configured to align a crown 430 to the surrounding anatomy. By providing an intermediate component, abutments according to the present embodiments can provide for the placement of a crown 430 at different angles and positions by making changes to the abutment 427 without requiring replacement of the implant 413. The abutment 427 may be customized based on a post-healing model (produced via post-healing imaging), or it can be customized based on the initial or an intermediate imaging.

FIGS. 6A, 6B, 6C, and 6D illustrate an implant embodiment that has one side that is configured to permit and promote osseointegration, thereby forming a thicker layer of bone adjacent the tooth. The implant can be used in conjunction with the offset abutment and an implant location positioned remotely from the natural axis of the socket, as described with reference to FIGS. 5A and 5B. Here, a natural tooth root 602 is shown in axial section with bone 606 adjacent the buccal side of the tooth. An implant 604 is about the size of the natural root 602 and has a recessed portion 612 in the buccal face thereof. Standoffs 610 can be attached to the recess portion to help position the axis of the implant in the desired position and maintain spaces where bone can grow to help support the implant 604. The standoffs 610 may be fabricated of a material that permits and/or promotes osseointegration. Referring now also to FIG. 6C, the bone is shown after having grown into the recess portion 612 and integrated into the standoffs 610. For example, the standoffs 610 can be made from resorbing material (e.g., coral) or from metal foam. Alternatively, they may be integral portions of the implant. In embodiments, the standoffs are elongate members parallel to the axis of the tooth rather than low aspect-ratio studs as depicted at 610. Other variations are also possible.

FIG. 7 illustrates an implant with a through-hole for anchoring an implant. An implant 139 as described above with reference to FIG. 1C has a through hole 702 formed in its side. A model of the through hole 702 may be digitally formed in the 3D model and fabricated as part of the casting or milling process as described above. One or more through holes may be formed. In an exemplary embodiment, a single hole of 4 mm in diameter, for example, may be provided, which is a size that will permit ingrowth of bone. After fabrication of the implant, the one or more through-hole(s) 702 may be fully or partly filled with scaffold or osteogenic materials prior to implantation. Where multiple through-holes are provided, such holes may have crossing axes, for example, or parallel ones.

According to the teachings of the present specification, implants can be fabricated using conventional equipment. In an embodiment, however, software may be provided which performs the function of accepting data representing the true shape and size of a furcated natural root and modifies the structure to eliminate or reduce the furcation. The software may generate an output in the form of a three-dimensional model which may then be processed by external software that generates control instructions for a milling machine or other computer controlled fabrication device or system such as a three-dimensional printer, photopolymerization-based fabrication as used for rapid prototyping, or computer aided machining. So a system for implementing may be provided using existing imaging technology to provide multiple images to create a three-dimensional model and then to permit the model to be modified as taught. Existing fabrication technology may be used to control fabricating system based on the modified model. Modification of the three-dimensional model may be done manually. The data of the modified model may be stored in a data store such as a random access memory, a nonvolatile data storage device such as a rotating disk or flash memory or it may be transmitted through a data channel to a remote computer that receives data for fabrication purposes, such as one at a dental laboratory.

In an alternative embodiment, to create an implant with a reduced or eliminated furcation, which is otherwise a copy of the natural root, a casting representing the shape of the root may first be made from the socket or an extracted root or tooth. Then the casting may be manually modified to eliminate or reduce the furcation by adding a material to fill in the recess. Alternatively, the furcated casting could be inserted in a tight elastic “sock” and liquid curable resin injected into it so that concavities are filled in. The curable resin may then be cured leaving a modified model. The model may then be used to create a titanium or other casting according to known techniques.

An implant may be fabricated by other means as well. The implant of any of the embodiments may be customized such that the surface details of a natural tooth root are represented in the implant with sub-millimeter precision. Thus, an implant of the disclosed embodiments may be a naturally shaped implant with a solid body having a shape that conforms precisely to a uniquely-shaped furcated portion of a natural tooth root of a unique living patient. The surface details of the implant may be preserved, with the tooth root having a recess that defines a furcation of the tooth root, the tooth root also having an external portion along a same axial extent of the tooth root as the recess, except that the furcation of the implant has a recess that is substantially reduced relative to the tooth root, or completely absent.

In addition, aspects of the disclosed subject matter assist with acquisition of X-ray images at specific, pre-set aiming directions for radiographically imaging a target, such as a tooth and/or multiple teeth, or any anatomical feature of the mouth. Embodiments include devices that assist a dental clinician in establishing predefined aiming directions of a target that are reproducible and define multiple aiming directions. The devices may be made so as to be autoclavable or disposable. In addition, or alternatively, embodiments include devices that may be attached to, and used with, currently existing bitewing bit block supports.

Present embodiments may form components of a system in conjunction with a computer system and software to produce three-dimensional representations based upon radiographs of multiple angles. Multiple-angle images, for example, can be combined to create a three-dimensional model, and the model used to enable three-dimensional computer-aided fabrication of a dental prosthetic based on the model.

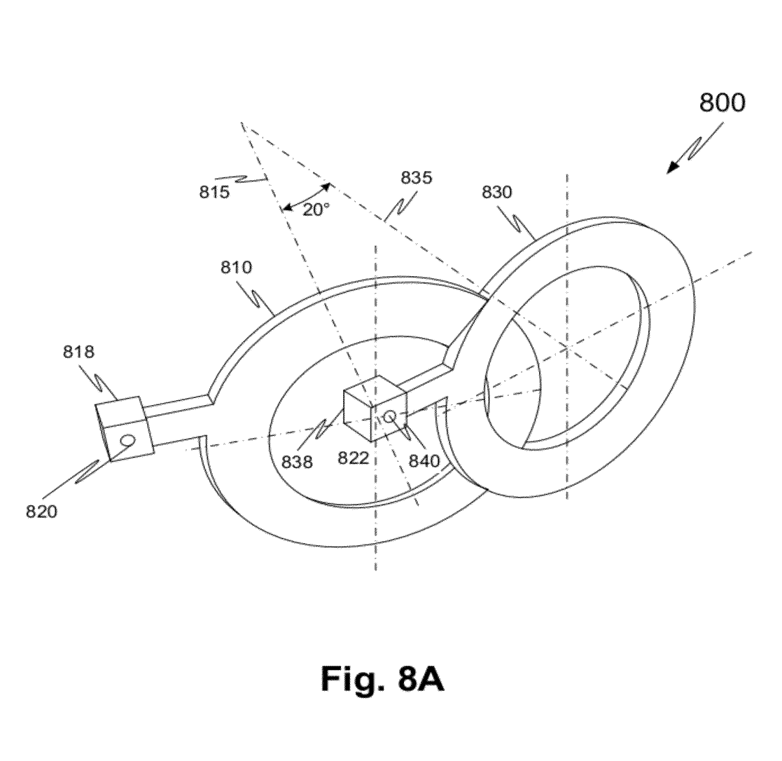

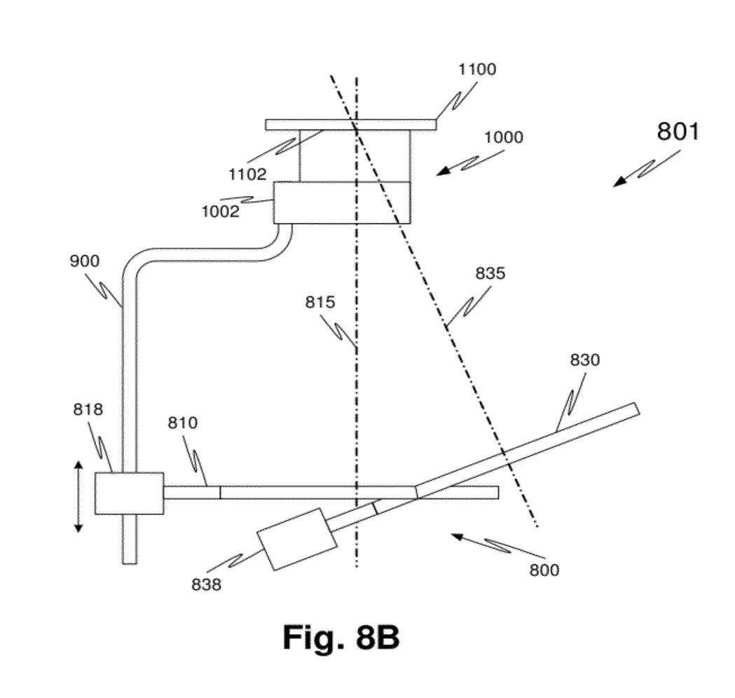

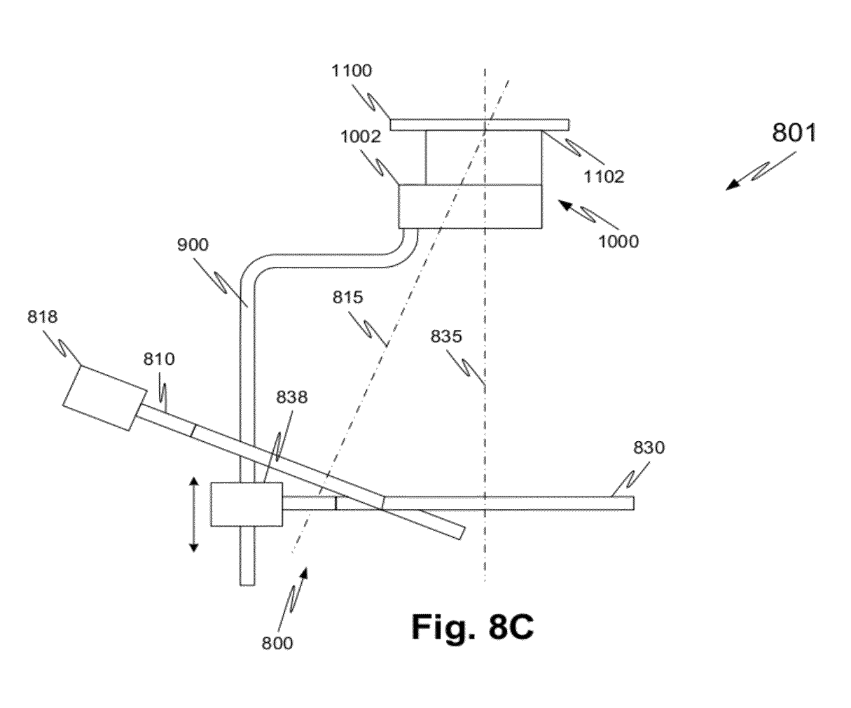

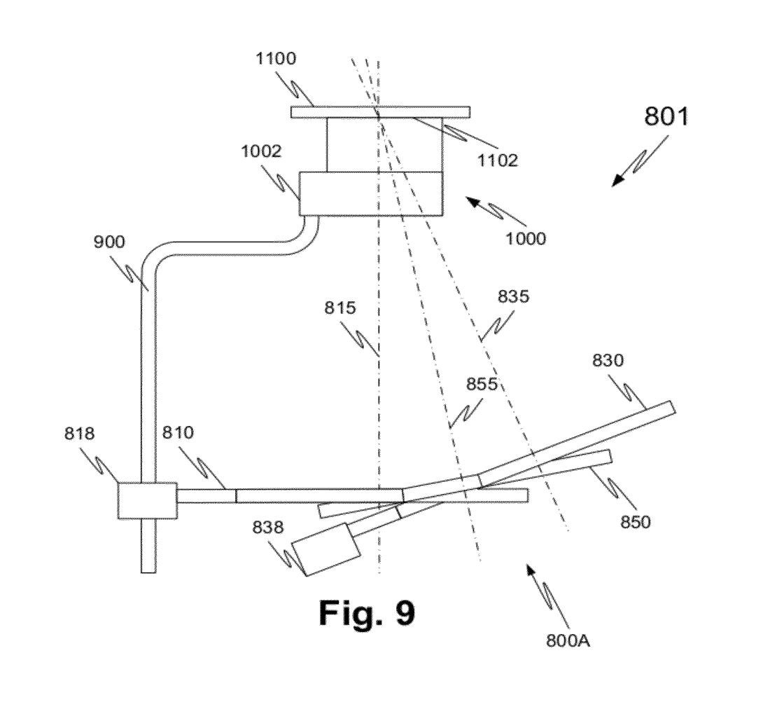

Referring to FIGS. 8A-8C, an aiming apparatus 800 is configured to assist alignment of a tube head of an X-ray apparatus such that X-ray beams are directed through a target at an image receptor at angles defined by the aiming apparatus 800. For example, the target may be a tooth or other animal anatomy. The angles may be chosen to provide sufficient information for three-dimensional modeling from the resulting planar projections of the target. The angles may be chosen also to minimize occultation (for example by adjacent anatomy such as an adjacent tooth or root) and may be chosen to be sufficient for the particular geometry. The aiming apparatus 800 may be formed of a material that is relatively transparent to X-rays, such as a non-metallic material, for example, any of various suitable thermoplastics.

The aiming apparatus 800 may be formed of a single integral structure using techniques such as injection molding. The aiming apparatus 800 may be formed in a single molding operation or formed from multiple elements that attached together to form a composite structure. The aiming apparatus 800 may be autoclavable, reusable, or disposable. Mechanically, the aiming apparatus 800 may be “universal” in the sense that it can be configured to be compatible with other existing radiography instruments in the market, such as support arms, bitewing bite blocks, image receptors, and/or X-ray tube heads.

The aiming apparatus 800 includes a first ring 810 and a second ring 830. The first ring 810 and the second ring 830 can be arranged as shown in FIG. 8A. The rings 810, 830 may be substantially the same shape and size. The first ring 810 may overlap the second ring 830 as shown. The positions and angles of the rings 810, 830 are such that, when positioned on a bitewing bite block support (See FIG. 8B), the aiming axes 815, 835 intersect approximately at an image plane 1102 of a radiographic imaging device 1100.

In the embodiment shown in FIG. 8A, first ring 810 has a body portion that defines an angle and position such that the X-ray tube head is aimed at a target. Projecting from the first ring is a receiving member 818 which includes an aperture 820 through which a support arm 900 can be inserted. The position of the receiving member 818 relative to the first ring 810 may be based on the configuration of a support arm 900, an image receptor 1102, and/or a bite block device 1000 with which the aiming apparatus 800 is to be coupled.

The receiving member 818 is configured to receive a support arm 900 of an image receptor 1100/bite block device 1000 (not shown in FIG. 8A). The support arm 900 can be inserted through aperture 820, and the aiming apparatus 800 can be moved along the length of the support arm 900 to a desired position. Markings and/or detents may be provided on the aperture 820/support arm 900 combination to assist with positioning and/or retention of the aiming apparatus 800. The aperture 820 can have any suitable shape in section, such as a square or other polygon. The ring 810 may be held in position by friction or by locking engagement with the support arm 900.

The second ring 830 can be configured similar to the first ring 810 and attached to define an angle with respect to the first ring 810, for example, a separation angle of twenty degrees. The receiving member 838 with aperture 840 projects from the second ring 830 and is configured to receive a support arm 900 of an image receptor 1100/bite block member 1000. As with the first ring 810, the support arm 900 can be inserted through aperture 840 of the second ring 830, and the aiming apparatus 800 can be moved along the length of the support arm 900 to a desired position closer or further from the image plane 1102 to accommodate a patient's anatomy. Note that receiving member 838 may overlap an open inner portion 822 of the first ring 810 such that access to aperture 840 is substantially unobstructed by the body of the first ring 810. Markings and/or detents may be provided on the aperture 840/support arm 900 combination to assist with positioning and/or retention of the aiming apparatus 800. Other positioning devices may also be used such as a linear positioner, slide-lock device, pantograph, or other device.

The rings 810, 830 are offset and canted with respect to each other such that the axes 815, 835 centered at openings of the rings intersect at the plane 1102 of the image receptor 1100. The rings 810, 830 may be canted with respect to one another at angles predetermined to permit the creation of a three-dimensional representation. For example, the rings 810, 830 may be angled from above zero degrees to twenty-two degrees. Or the rings 810, 830 may be angled with respect to one another from eleven to twenty-two degrees. Or they may be canted with respect to one another at an angle from ten to twenty degrees. The rings 810, 830 in FIG. 8A, for example, are angled at twenty degrees with respect to one another.

When the aiming apparatus 800 is coupled to a support arm 900 affixed to a bite block device 1000 and an image receptor 1100, the clinician can aim an X-ray apparatus, using the arrangement of the rings 810, 830 to take radiographs at the angles defined by the ring positions. Thus, radiographs can be taken at predetermined, reproducible angles without requiring the clinician to move or otherwise reposition the aiming apparatus 800.

Either of the rings 810, 830 can be arranged such that a respective axis 815, 835, which passes through the center of the ring 810, 830 forms an orthogonal or substantially orthogonal angle with respect to a receiving surface 1102 of an image receptor 1100. Consequently, the axis of the other of the rings forms an oblique angle with respect to the receiving surface 1102 of the image receptor 1100. In another embodiment, the axes of the rings are both oblique to the image plane, for example, forming equal and opposite angles of the normal.

FIG. 8B is an overhead view of a radiographic system 801 with aiming device 800, a bite wing or block device 1000 coupled to the aiming apparatus 800 via a support arm 900, and an image receptor 1100 coupled to bite block device 1000. In FIG. 8B, the aiming apparatus 800 is coupled in a first position to bite block device 1000 and image receptor device 1100.

The bite block device 1000 can include a receiving portion 1002 to receive support arm 900. Alternatively, the support arm 900 can be integral, or permanently attached to the bitewing bite block device 1000. Though FIG. 8B shows support arm 900 being coupled to the receiving portion 1002 on the left side of the bitewing bite block device 1000 (in plan view), the receiving portion 1002 can be arranged on the right side of the bitewing bite block device 1000 (in plan view) or arranged below or above the bitewing bite block device 1000. In alternative embodiments, a plurality of receiving portions 1002 may be provided, each located at a different respective position (aiming angle) relative to the bitewing bite block device 900 to permit the selection of different positions and angles. The particular arrangement of one or more of the receiving portions 1002 may be compatible with different shapes, sizes, and/or configurations of support arms 900. The bitewing bite block device 1000 also may be configured with a base or stabilizer (not shown) to hold against an opposite tooth row. Such a configuration can stabilize the bitewing bite block device 1000 in the patient's mouth during imaging.

The image receptor 1100 is coupled to the bitewing bite block device 1000. The image receptor 1100 may be a holder for supporting radiographic film or a variety of digital sensors to create the image on a digital medium or memory (not shown). A digital-type image receptor may be configured to permit multiple radiographs to be taken without having to open the patient's mouth to gain access to the used film and to insert a new film element.

Support arm 900 can be coupled to bitewing bite block device 1000 via receiving portion 1002. Support arm 900 is configured to slidably engage receiving members 818, 838 of the rings 810, 830. FIG. 8B, for example, shows support arm 900 slidably engaged with receiving member 818 of first ring 810. Receiving members 818, 838 of the rings 810, 830 may be moved or slid along the length of the support arm 900 to position the aiming apparatus 800. The arm member 900 may have markings and/or detents (not shown) to assist with positioning and/or retention of the aiming apparatus 800. In various embodiments, the markings and/or detents can be based on the particular angular configuration of the rings 810, 830. For example, the markings and/or detents may indicate a position along the support arm 900 at which to position the aiming apparatus 800 so that the axes 815, 835 of the rings cross at the image plane 1102. The markings and/or detents also can be arranged to take into consideration the particular configuration of the bite block device 1000, the image receptor 1100, and/or the aiming apparatus 800.

In any of the support arm 900 embodiments disclosed herein, the support arm 900 may have a non-round cross-section to prevent rotation of the supported aiming apparatus 800 (or other similar embodiments disclosed herein). The aiming apparatus 800 may have receiving apertures 820, 840 that have a shape that engages with the cross-sectional shape of the support arm 900 such that rotation or pivoting about the support arm 900 is prevented. In addition a depth of the apertures 820, 840 (for example, a depth of the receiving member 818) may be such that it holds the aiming apparatus 800 at a precise orientation. Alternatively, a tight frictional engagement may be provided such the aiming apparatus 800 is firmly held to maintain a predefined orientation. The structure may be such that the aiming directions reliably cross as illustrated in FIG. 8B when the aiming apparatus 800 is attached to the support arm 900.

The aiming apparatus 800 shown in FIG. 8B is arranged in a “first” position, such that receiving member 818 of the first ring 810 is coupled to support arm 900 and such that receiving member 838 of the second ring 830 is free. In this configuration, the axis 815, at the center of the first ring 810, can be orthogonal to, or substantially orthogonal to, receiving surface 1102 of the image receptor 1100. The axis 815 may be directly in the center of the receiving surface 1102, or it may be offset therefrom. Similarly, when the support arm 900 is inserted in the receiving member 838, the axis 835, at the center of the second ring 830, can define an aiming axis that is orthogonal to the receiving surface 1102.

In operation, a clinician takes a first radiograph, with the X-ray head of the X-ray apparatus brought into position and aligned with the first ring 810 (manually by the clinician or otherwise) to output X-rays toward the object or objects to be imaged and the image receptor 1100, based on the alignment of the first ring 810. The bite block device 1000 may be configured automatically to align the support arm 900 such that the aiming directions of the rings 810, 830 are established when the patient bites down. After the first radiograph, the X-ray head of the X-ray apparatus can be moved and aligned with the second ring 830 (manually by the clinician or otherwise) to output X-rays toward the object or objects to be imaged and the image receptor 1100, based on the alignment of the second ring 830. The order of use of the rings 810 and 830 may be reversed. An electronic system can associate the angle (including the orthogonal angle) at which each radiograph was taken. This information may be stored electronically for use in generating a three-dimensional representation.

FIG. 8C is an overhead view of the radiographic system 801 in FIG. 8B, with the aiming apparatus 800 attached to the support arm 900 via the receiving member 838. In the illustrated position, the receiving member 818 is free. In this configuration, the axis 835 associated of the second ring 830 is orthogonal to or substantially orthogonal to receiving surface 1102 of the image receptor 1100. In this second position, the axis 815 at the center of the first ring 810 is at a non-orthogonal angle with respect to the receiving surface 1102.

FIG. 9 is an overhead view of a radiographic system 801 having an aiming apparatus 800A coupled to the bite block device 1000 and image receptor 1100. Like the aiming apparatus 800 discussed above for FIGS. 8A-80, the aiming apparatus 800A in FIG. 9 is configured to assist with alignment of a tube head of an X-ray apparatus such that X-ray beams output therefrom are directed through a target at an image receptor 1100. The aiming apparatus 800A in FIG. 9 is similar to the aiming apparatus 800, but has an additional, “middle” ring 850 connected to the first 810 and second 830 rings. The rings 810, 830, 850 of the aiming apparatus 800A can be substantially the same size and generally of the same shape, or can have different sizes and/or shapes. The rings 810, 830, 850 also can include indicia indicating an order of use. Portions of the rings 810, 830, 850 may overlap others of the rings. Since each of the rings 810, 830, 850 is made of a material that is transparent to X-rays, the overlapping will have little or no effect on images produced.

The middle ring 850 can define an aiming direction 855 that is between the directions 815 and 835 of the first and second rings 810 and 830. In various embodiments, the aiming direction 855 may bisect the angle between the extreme aiming directions 815 and 835. For example, the extreme rings 810, 830 may define aiming directions that are between about ten and twenty degrees apart. The aiming directions 815, 835, 855 may be selected so that once the aiming apparatus 800A is coupled to a support arm 900 the clinician can aim an X-ray apparatus using the arrangement of the rings to take radiographs at the respective different angles. The radiographs may be taken at predetermined, reproducible angles and the clinician does not have to move or otherwise reposition the aiming apparatus 800A to take radiographs at different angles.

Either of the outer rings 810 or 830 can be arranged (i.e., coupled to support arm 900) such that an axis 815, 835, 855 passing through the center of a respective ring 810, 830, 850 forms any desired angle, such as orthogonal or substantially orthogonal, with respect to a receiving surface 1102 of an image receptor 1100. Consequently, the axes of the other of the outer rings and the aiming direction 855 of the middle ring 850 may be perpendicular or oblique to the receiving surface 1102. In other embodiments, the aiming directions defined by the rings 815, 835, 855 are all oblique to the receiving surface 1102.

In use, a clinician takes radiographs using each of the defined aiming axes 815, 835, and 855. In any of the embodiments disclosed, the image data may be stored along with information indicating the respective aiming directions using any suitable means.

The aiming apparatuses 800, 800A discussed above have been described as being arranged in a “horizontal” orientation (i.e., radiographs are taken at angles displaced from each other in a general horizontal direction). However, in alternative embodiments, aiming apparatuses similar to 800, 800A of FIGS. 8 and 9 also may be configured to define separation angles that lie in planes that contain (or are parallel to) a root axis of a target tooth. The aiming directions defined thereby may form any chosen angle with respect to the receiving surface 1102.

Embodiments of the disclosed subject matter also include an aiming apparatus that can provide for both vertical and horizontal orientations simultaneously. Such embodiments can include an aiming apparatus with four rings, for example, where a first two of the rings are displaced in a plane normal to the root axis and a second two are displaced, relative to the first two, by an angle lying in a plane parallel to the root axis. Alternatively, three rings may be provided, with a first ring displaced by angles lying in each of these planes from a respective second and third ring.

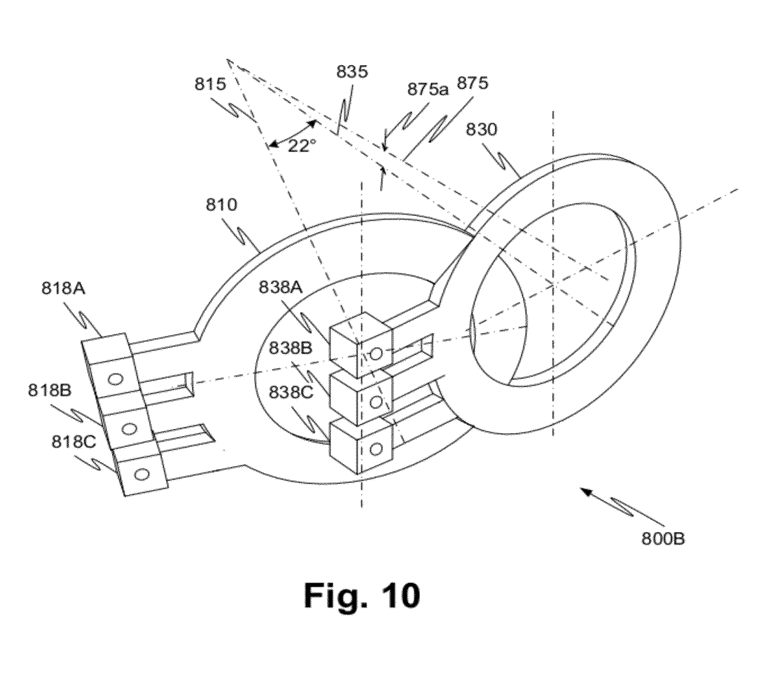

FIG. 10 illustrates an embodiment of an aiming apparatus 8008 for a radiographic system. The aiming apparatus 800B is similar to the aiming apparatus 800 in FIG. 8, but each of the rings 810, 830 includes a plurality of receiving members 818A-818C, 838A-838C for coupling to a support arm 900, thereby providing an ability to select a different position. The receiving members 818A-818C, 838A-838C may also define aiming directions that are displaced in terms the angle, the position, or both. For example, the aiming direction 875, which differs from aiming direction 835 by an angle 875a in a vertical plane (from the perspective of the drawing) could be defined by ring 830 when the arm is repositioned from receiving member 838A to receiving member 838B. The aiming directions may differ similarly for the other receiving members. Also, the different receiving members may reposition the target point so that the bite block can be moved for taking images of lower jaw anatomy or upper jaw anatomy.

Though FIG. 10 shows each ring 810, 830 having sets of three receiving members (818A-818C, for example), any suitable number of receiving members can be provided, and they can be arranged to project from any suitable position of the ring. Multiple receiving members are provide for one or more of the rings so the aiming apparatus 800B can be used with a variety of different configurations of bite block devices 1000, image receptors 1100, and/or support arms 900.

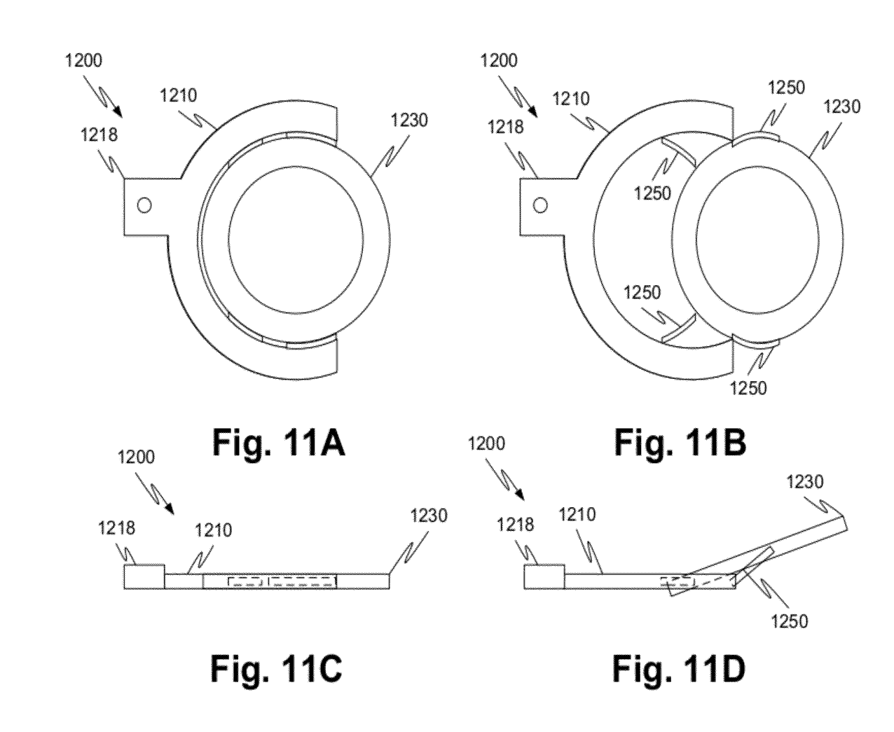

FIGS. 11A-11D illustrate an aiming apparatus 1200 for radiography according to yet another embodiment. FIGS. 11A and 11C show front and top views of the aiming apparatus 1200 in a first, non-extended position defining a first aiming direction. The ring 1230 can be extended, by way of articulation members 1250, to define an angle with respect to the first “ring” 1210. FIGS. 11B and 11D show the aiming apparatus 1200 in a second, extended position defining a second aiming direction. As in previous embodiments, the aiming angles may be determined for purposes of forming three-dimensional models from suitable planar projections. The two positions may be configured to be locking positions so that, for example, by means of a detent or spring mechanism, the positions of FIGS. 11C and 11D are indicated, respectively, by haptic feedback or arrived at without being at rest in any intermediate position. The aiming apparatus 1200 also can have a receiving member 1218 for coupling to a biteblock and/or an image receptor via an arm, for example.

Other embodiments of the disclosed subject matter include single-ring aiming apparatuses. Single-ring aiming apparatuses according to embodiments of the disclosed subject matter can be moveable in two directions to take radiographs at orthogonal angles with respect to an image plane 1102 as well as to take radiographs at non-orthogonal angles. For example, the ring may be rotatable and moveable linearly (i.e., from side to side) about a linear guide. The linear guide may have markings to ensure that a base member to which the ring is coupled is moved to the correct location, depending upon the rotation of the ring. The linear guide also may have detents to lock temporarily the ring. In another example, the ring may be rotatable and moveable along an arc-like pattern to take radiographs at orthogonal and non-orthogonal angles with respect to an image plane 1102.

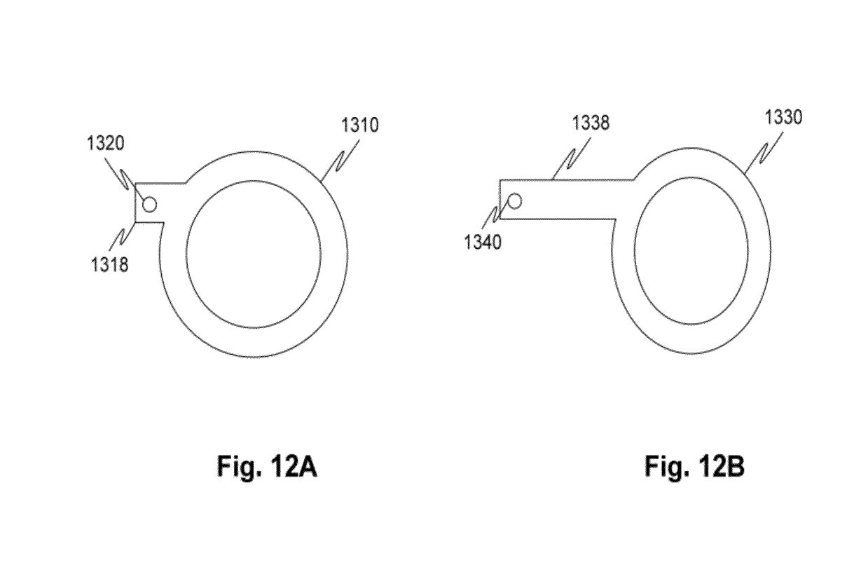

FIGS. 12A and 12B show a plurality of single ring aiming apparatuses 1310, 1330, as part of a kit. Each ring 1310, 1330 has a receiving portion 1318, 1338 with an aperture 13620, 1340 for coupling to an arm member 900. The kit may come with a housing that houses one or more sets of aiming apparatuses. Each aiming apparatus 1310, 1330 defines a respective aiming direction.

Though the foregoing disclosure has used the term “ring” to describe the general configuration of the aiming or aligning portion of the aiming apparatus, the disclosed subject matter described herein are not limited to rings. For example, the aiming or aligning portions of the aiming apparatus may be arc-shaped, half-circle-shaped (e.g., first ring 1210 in FIGS. 11A-11C), x-shaped, etc.

In all the aiming device embodiments, the aiming device (for example, the structure having two rings) may be provided with a support member adapted to fixedly position and orient the aiming device with respect to dental anatomy of a patient. For example, the aiming rings may be provided with a support boss with a hole. The hole may be shaped to receive a support arm extending from the bite block support. A polygonal or other non-round shape may be used to prevent rotation about a correspondingly shaped support arm. In any of the embodiments, aiming rings may be replaced with position and direction indicators of other shapes. For example, U-shaped indicators or polygon shaped indicators may be used. Another alternative is a transparent member with a position indication printed thereon, for example a gunsight (e.g., crosshairs) pattern or circle.

Having now described embodiments of the disclosed subject matter, it should be apparent to those skilled in the art that the foregoing is merely illustrative and not limiting, having been presented by way of example only. Numerous modifications and other embodiments (e.g., combinations, rearrangements, etc.) are within the scope of one of ordinary skill in the art and are contemplated as falling within the scope of the disclosed subject matter and any equivalent thereto. It can be appreciated that variations to the present disclosed subject matter would be readily apparent to those skilled in the art, and the present disclosed subject matter is intended to include those alternatives. Further, since numerous modifications will readily occur to those skilled in the art, it is not desired to limit the disclosed subject matter to the exact construction and operation illustrated and described, and accordingly, all suitable modifications and equivalents may be resorted to, falling within the scope of the disclosed subject matter.

1. A computer readable medium containing executable program instructions for performing a method for making a dental implant, the method comprising:
accepting data representing a true shape and size of a furcated natural root of a tooth having a recess between the branches of the root furcation;
creating a three-dimensional model from the accepted data, where the three-dimensional model has a true shape of the furcated natural root except for a reduced size of the recess; and
generating data for controlling a machine to fabricate the dental implant responsively to the three-dimensional model or data representing the three-dimensional model.

2. The medium of claim 1, the method further comprising outputting the data for controlling the machine to a data store or a communications channel.

3. The medium of claim 1, wherein the creating a three-dimensional model is such that the three-dimensional model has a true shape of the furcated natural root except for an absence of the recess.

4. The medium of claim 1, wherein the creating the three-dimensional model includes modifying the three-dimensional model to reduce the size of the recess.

5. The medium of claim 1, wherein the accepting includes storing a set of coordinates representing a geometry of a surface of a portion of the furcated natural root in a data store.

6. The medium of claim 1, wherein the dental implant is shaped so that sides that surround an axis thereof are faithful to the shape of the furcated natural root at axial positions toward an occlusal end of the tooth to the axial position of the furcation but the implant is substantially less furcated than the furcated natural root.

7. The medium of claim 1, wherein the creating a three-dimensional model includes applying a surface-fit spline algorithm with a minimum smoothness constraint to an intermediate three-dimensional model to produce a smoother three-dimensional model, the method further comprising generating instructions to control the machine to fabricate the dental implant responsively to the smoother three-dimensional model and outputting the instructions.

8. The medium of claim 1, wherein the creating a three-dimensional model from the accepted data includes modifying a depth of a concavity in a side thereof facing approximately away from a central longitudinal axis thereof.

9. A method for making a dental implant, comprising:
creating a first three-dimensional model having a true shape and size of a furcated natural root having a void between the branches of the root furcation;
modifying the first three-dimensional model resulting in a second three-dimensional model having a true shape of the furcated natural root except for a reduced size of the void; and
fabricating the dental implant based on the second three-dimensional model.

10. The method of claim 9, wherein the creating includes making a casting from an empty tooth socket of a living patient.

11. The method of claim 9, wherein the creating includes acquiring image data representing the furcated natural root and calculating the first three-dimensional model from the image data.

12. The method of claim 9, wherein the creating includes making a casting from a tooth socket.

13. The method of claim 9, wherein the fabricating includes machining a physical model and casting titanium responsively to the physical model.

14. The method of claim 9, wherein the modifying is such that the dental implant is shaped so that those of its sides surrounding a central longitudinal axis thereof are true to the shape of the furcated natural root but so that an end portion thereof is substantially less furcated than the furcated natural root.

15. The method of claim 9, wherein the modifying includes modifying a depth of a concavity in a surface of the first three-dimensional model which has a normal that is perpendicular to a central longitudinal axis of the first three-dimensional model.

16-29. (canceled)

30. A device for creating radiographic images, comprising:
an aiming member having at least two direction indicators configured to indicate first and second aiming axes, the first and second aiming axes being separated from one another by a predetermined angle;
the aiming member having a support member adapted to fixedly position and orient the aiming member with respect to dental anatomy of a patient.

31. A device according to claim 30, wherein the support member includes a boss having a polygonal hole configured to receive a support arm.

32. A device according to claim 30, wherein the at least two direction indicators include overlapping rings that are mutually offset and lying in respective planes offset by the angular separation between the two aiming axes.

33. A device according to claim 30, wherein the support member includes a support arm and a bitewing block.

34-56. (canceled)

57. A device according to claim 30, wherein the predetermined angle is between 10 and 22 degrees
